(19) United States
(12) Patent Application Publication
(10) Pub. No.: US 2006/0051852 A1
(43) Pub. Date: Mar. 9, 2006

(54) Apyrase polypeptides and nucleic acids encoding same

(76) Inventors: Thomas M. Smith, Weymouth, MA (US); Terence L. Kirley, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 10/498,691

(22) PCT Filed: Dec. 12, 2002
(86) PCT No.: PCT/US02/39643

§ 371 Date: Apr. 18, 2005

Related U.S. Application Data
(60) Provisional application No. 60/340,976, filed on Dec. 12, 2001.

(51) Int. Cl. C12N 9/02; C07H 21/04; C12P 21/06

(52) U.S. Cl. 435/189; 435/69.1; 435/320.1; 435/325; 536/23.2

(57) ABSTRACT

The present invention provides novel isolated polynucleotides encoding a novel extracellular nucleotidase, which belngs to a new family of calcium dependent apyrases. Also provided are antibodies that immunospecifically bind to the apyrase polypeptide or any derivative (including fusion derivative), variant, mutant or fragment of the novel polypeptide, polynucleotide or antibody. The invention additionally provides methods in which the apyrase polypeptide, polynucleotide and antibody are utilized in the treatment of vascular disorders, including disorders characterized by platelet aggregation.

This invention was made with government support under Grant No. NIH R01 HL59915-01A2 awarded by the National Institutes of Health. The government has certain rights in the invention.

### FIELD OF THE INVENTION

The invention relates generally to nucleic acids and polypeptides, and more specifically to nucleic acids and polypeptides encoding apyrase enzymes, as well as vectors, host cells, antibodies and recombinant methods for producing the polypeptides and polynucleotides.

### BACKGROUND OF THE INVENTION

Ecto-apyrases or ATP-diphosphohydrolases (EC 3.6.1.5) are a class of glycosylated extracellular enzymes that hydrolyze extracellular ATP to AMP as follows:
ATP+H₂O→ADP+Pᵢ
ADP+H₂O→AMP+Pᵢ

Apyrases have been implicated in the maintenance of hemostasis and inhibition of platelet aggregation through the hydrolysis of extracellular adenosine diphosphate. Apyrases can be found among the larger family of extracellular nucleotidases formerly referred to as the E-type ATPases (now renamed E-NTPDases—ectonucleoside triphosphate diphosphohydrolases. At least six different members of the human E-NTPDases have been discovered, each of which possesses different enzymatic properties and different physiological localizations.

In spite of the broad distribution of the different apyrases, each enzyme usually has the following general characteristics: (1) a nucleotide hydrolysis site situated on the exterior of the cell, (2) a strict dependency upon divalent cations (usually calcium or magnesium) for activity, (3) an ability to rapidly hydrolyze a wide range of extracellular nucleoside tri- and diphosphates in addition to ATP and ADP, at turnover rates approaching that of acetylcholinesterase, and (4) extended amino acid sequence homology in the extracellular region of the proteins, with five “apyrase conserved regions” shown to be essential for enzymatic activity. Reported members of the E-NTPDases possess these conserved phosphate-binding motifs consisting of invariant amino acids comprising the nucleotide binding and hydrolysis site.

Functionally, extracellular apyrases have been implicated in playing roles in the cardiovascular system. Apyrases of the circulatory system have been reported to be important in the maintenance of blood fluidity. For example, the endothelial cell plasma membrane apyrase CD39 (renamed E-NTPDase-1) has been implicated in the maintenance of normal blood fluidity through its hydrolysis of extracellular adenosine diphosphate (ADP). At the site of vascular injury, nucleotides such as ADP are released by activated platelets and are one of the most important physiological agonists of subsequent platelet recruitment, aggregation and plug formation. This vascular enzyme E-NTPDase-1 is believed to play an important role in thromboregulation through the hydrolysis of excess nucleotide tri- and diphosphates, thus keeping the homeostatic process tightly regulated to prevent excess clot formation and vessel occlusion. Extracellular apyrases are thus considered important for the termination of purinergic receptor-mediated responses, including those of platelet activation and aggregation, and have recently been proposed to be one of the three major systems (including the eicosanoids and nitric oxide) responsible for the maintenance of hemostasis and thromboregulation.

### SUMMARY OF THE INVENTION

The invention is based, in part, upon the discovery of a human polynucleotide sequence encoding a new soluble apyrase. The polypeptide encoded by the cDNA is related to the Cimex lectularius apyrase. The encoded polypeptide defines a new family of extracellular apyrases. The nucleic acids and polypeptides according to the invention disclosed herein are referred to as APYR nucleic acids and polypeptides, respectively.

In one aspect, the invention provides a purified polypeptide that includes an amino acid sequence at least 80% identical to amino acids 39-371 of SEQ ID NO:2. In some embodiments, the polypeptide hydrolyzes adenosine triphosphate (ATP) or adenosine diphosphate (ADP), or both.

In some embodiments, the ATP or ADP hydrolytic activity of the polypeptide is calcium-dependent.

In some embodiments, the ATP or ADP hydrolytic activity of the polypeptide is insensitive to sodium azide.

In some embodiments, the polypeptide includes at least one of the amino acid sequences IADLD (SEQ ID NO:3), DDRTG (SEQ ID NO:4), PWVIL (SEQ ID NO:5), GFKAEW (SEQ ID NO:6), FLPR (SEQ ID NO:7), NIGF (SEQ ID NO:8), and KYEG (SEQ ID NO:9). More preferably, the polypeptide includes at least two (such as IADLD (SEQ ID NO:3) and KYEG (SEQ ID NO:9). Most preferably, the polypeptide includes three, four, five, six, or even seven of these sequences.

A preferred polypeptide is an amino acid sequence at least 95% identical to amino acids 39-371 of SEQ ID NO:2, and which shows calcium-dependent and azide-insensitive insensitive hydrolysis of adenosine triphosphate (ATP) or adenosine diphosphate (ADP). The polypeptide in addition includes at least three of the amino acid sequences IADLD (SEQ ID NO:3), DDRTG (SEQ ID NO:4), PWVIL (SEQ ID NO:5), GFKAEW (SEQ ID NO:6), FLPR (SEQ ID NO:7), NIGF (SEQ ID NO:8), and KYEG (SEQ ID NO:9). In preferred embodiments, the polypeptide includes amino acids 39-371 of SEQ ID NO:2, e.g., the polypeptide may consist of amino acids 39-371 of SEQ ID NO:2.

In another aspect, the invention provides an isolated nucleic acid molecule encoding an APYR polypeptide. In preferred embodiments, polypeptide comprising an amino acid sequence at least 80% identical to amino acids 39-371 of SEQ ID NO:2. In some embodiments, the encoded polypeptide hydrolyzes adenosine trinucleotide phosphate (ATP) or adenosine dinucleotide phosphate (ADP), or both.

In some embodiments, the ATP or ADP hydrolytic activity of the encoded polypeptide is calcium-dependent.

In some embodiments, the ATP or ADP hydrolytic activity of the encoded polypeptide is insensitive to sodium azide.

In some embodiments, the encoded polypeptide includes at least one of the amino acid sequences IADLD (SEQ ID NO:3), DDRTG (SEQ ID NO:4), PWVIL (SEQ ID NO:5), GFKAEW (SEQ ID NO:6), FLPR (SEQ ID NO:7), NIGF (SEQ ID NO:8), and KYEG (SEQ ID NO:9). More preferably, the encoded polypeptide includes at least two sequences (such as IADLD (SEQ ID NO:3) and KYEG (SEQ ID NO:9). Most preferably, the polypeptide includes three, four, five, six, or even seven of these sequences.

A preferred nucleic acid encodes a polypeptide that includes an amino acid sequence at least 95% identical to amino acids 39-371 of SEQ ID NO:2, and which shows calcium-dependent and azide-insensitive hydrolysis of adenosine triphosphate (ATP) or adenosine diphosphate (ADP). The encoded polypeptide in addition includes at least three of the amino acid sequences IADLD (SEQ ID NO:3), DDRTG (SEQ ID NO:4), PWVIL (SEQ ID NO:5), GFKAEW (SEQ ID NO:6), FLPR (SEQ ID NO:7), NIGF (SEQ ID NO:8), and KYEG (SEQ ID NO:9). In preferred embodiments, the encoded polypeptide includes amino acids 39-371 of SEQ ID NO:2, e.g., the nucleotide in some embodiments includes 606-1604 of SEQ ID NO:1.

The invention also includes an oligonucleotide that includes a portion of an APYR nucleic acid. For example, the oligonucleotide can be at least 10 nucleotides in length and include at least nine contiguous nucleotides of SEQ ID NO:1.

Also provided by the invention is a vector that includes an APYR nucleic acid. The vector can include, e.g., a nucleic acid encoding an APYR nucleic acid. In a further aspect, the invention includes a cell that includes the APYR-containing nucleic acid vector.

In a further aspect, the invention provides an antibody that selectively binds to an APYR polypeptide, e.g., a polypeptide that includes an amino acid sequence at least 80% identical to amino acids 39-371 of SEQ ID NO:2. The antibody can be a polyclonal antibody or a monoclonal antibody. In some embodiments, the antibody neutralizes ATP or ADP hydrolytic activity of an AYPR polypeptide.

Also within the invention is a pharmaceutical composition that includes an APYR polypeptide, an APYR nucleic acid (including a AYPR polypeptide encoding nucleic acid or an APYR anti-sense nucleic acid), or an APYR antibody.

The invention also includes a method of producing an APYR polypeptide by culturing a cell that includes an APYR-encoding nucleic acid under conditions allowing for expression of the polypeptide encoded by the APYR nucleic acid.

In a still further aspect, the invention includes a method for identifying a modulator of platelet aggregation by contacting a test agent with an APYR polypeptide (such as a polypeptide that includes amino acids 39-371 of SEQ ID NO:2) and determining whether the test agent binds specifically to the polypeptide. Specific binding of the agent to the APYR polypeptide indicates the agent is a modulator of platelet aggregation.

Also provided by the invention is method of hydrolyzing ATP or ADP in a biological sample. The invention includes contacting the sample with an APYR polypeptide. The biological can include, for example, blood components (such as white blood cells, platelets, serum, plasma, vascular endothelial cells). The sample can be provided in vitro or in vivo.

The invention also includes a method of inhibiting platelet aggregation in a subject by introducing into the subject an agent that increases levels of an AYPR polypeptide of claim 1 in the subject.

The subject is preferably a mammal, such as a human, a non-human primate such as a chimpanzee, ape, or gorilla; a dog, cat, cow, horse, or pig.

In some embodiments, the agent is a polypeptide that includes amino acids 39-371 of SEQ ID NO:2. Other agents include a nucleic acid encoding amino acids 39-371 of SEQ ID NO:2 (such as nucleotides 606-1604 of SEQ ID NO: 1).

Also provided by the invention is a method of treating a thrombosis in a subject, the method comprising introducing into the subject an agent that increases levels of an APYR polypeptide in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIGS. 1A-1C are a representation of the nucleotide (SEQ ID NO:1) and encoded amino acid sequence (SEQ ID NO:2) of a human apyrase clone.

FIG. 2 is a graph showing apyrase activity secreted by COS-1 cells transfected with human apyrase cDNA.

FIG. 3 is a graph showing the results of size exclusion chromatography of soluble expressed human apyrase.

FIG. 4A is a dot-blot analysis showing tissue-specific expression of a soluble human apyrase mRNA

FIG. 4B is a representation showing relative expression levels of soluble human apyrase mRNA in various tissue types.

FIG. 5 is a CLUSTALW alignmnent of human apyrase with related polypeptide sequences.

FIG. 6 is a western blot showing the secreted apyrase protein before and after deglycosylation with peptide N-glycosidase F.

### DETAILED DESCRIPTION OF THE INVENTION

The invention provides nucleic acids encoding novel polypeptides with apyrase activity. Nucleic acids and polypeptides according to the invention are referred to herein as “APYR” nucleic acids and polypeptides.

The invention is based, in part, on the discovery of a 1.69 kilobase novel human apyrase cDNA clone. The cDNA clone includes an open reading frame encoding a 371 amino acid protein with one putative Asn-linked glycosylation site and several potential protein kinase phosphorylation sites. An amino-terminal cleavable signal peptide in the encoded polypeptide generates a secreted, soluble protein with a predicted molecular mass of 37,193 daltons.

Expression of the cDNA in mammalian COS-1 cells yields a soluble apyrase enzyme secreted into the cell media. The ADP and ATP nucleotide hydrolyzing activity of the encoded polypeptide is dependent upon calcium for activity, with no significant activity detected in the absence of calcium or in the presence of magnesium. Unlike previously described apyrases, this soluble apyrase is insensitive to sodium azide inhibition. Size exclusion chromatography reveals a monomeric enzyme of 34-37 kDa, consistent with the predicted cleaved molecular mass. Multiple tissue Northern analyses identifies the transcript in a wide range of human tissues, including testis, placenta, prostate, and lung. No traditional apyrase conserved regions or nucleotide-binding domains, such as those described in the eNTPDases (e.g. NTPDase-1 or CD39), have been identified in the encoded human enzyme, indicating the encoded polypeptide is a member of a new family of extracellular apyrases.

A novel APYR nucleic acid sequence according to the invention was identified using an amino acid sequence isolated from the blood-sucking bed bug Cimex lectularius (GenBank accession number AAD09177; (Valenzuela et al., J. Biol. Chem. 273:30583-90, 1998). The bed bug sequence was used as a query sequence to perform a TBLASTN search (Altschul et al., Nucleic Acids Res. 25: 3389-402, 1997) of the GenBank human EST databases. Several human EST clones were identified that showed strong identity to the bedbug apyrase. One of these clones, obtained from mRNA in female breast tumor tissue, was sequenced. DNA sequencing was performed by the DNA Core Facility of the Department of Molecular Genetics in the College of Medicine at the University of Cincinnati, using fluorescent dye automated sequencing. For subsequent transfection and expression in eukaryotic cells, the insert was excised from the pT7T3D-PAC plasmid using NotI and EcoRI restriction endonucleases and ligated into the 5.4 Kb pcDNA3 eukaryotic expression vector (Invitrogen).

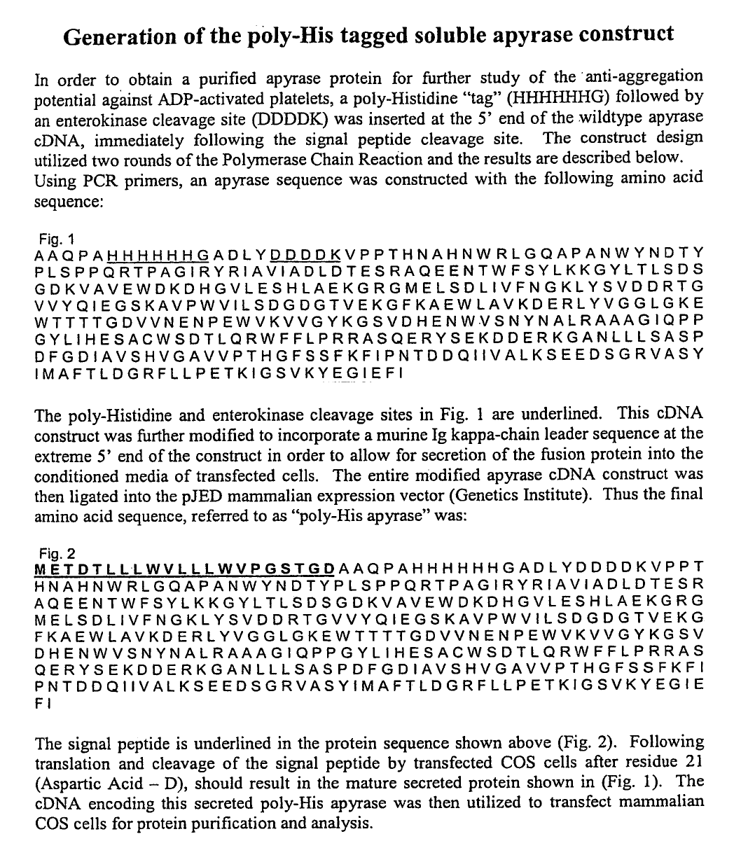

The sequence analysis reveals an insert size of 1691 bases. The cDNA sequence includes an open reading frame that extends from nucleotide 492 to 1604. The amino acid sequence deduced from this cDNA specifies a 371 amino acid protein (SEQ ID NO:11). The nucleotide and encoded amino acid sequence are shown in FIGS. 1A-1C. The apyrase clone includes 1691 nucleotides (SEQ ID NO:1). An open reading frame encoding a 371 amino acid protein (SEQ ID NO:2) is present. The initiation and termination codons are shown underlined in FIGS. 1A-1C. The initiation methionine is marked with a bold underline. An arrow (↑) represents the predicted signal peptide cleavage site. The N-linked glycosylation consensus sequence (NDTY) is underlined, and the putative Asn-glycan is indicated by an asterisk (*).

The PSORT II program predicts a cleavable signal peptide at residues 37, 38 (amino acids GR). Cleavage of this signal peptide on the carboxy-side of the Arg 38 residue results in a processed protein of 333 amino acids, with a calculated molecular mass of 37,193 Da and a pI of 5.13, assumring no post-translational modifications. No other cell targeting signals are detected in the sequence, indicating that the product of this gene is likely to be secreted outside the cells. The mature protein is predicted to have one N-glycosylation site (see Table 1), one cAMP- and cGMP-dependent protein kinase phosphorylation site, and six protein kinase C and casein kinase II phosphorylation sites, respectively.

Using the BLASTP search program provided by the National Center for Biotechnology Information (NCBI), the amino acid sequence of the human apyrase was used as a query sequence to examine the non-redundant protein database. In addition to the bedbug apyrase, several additional proteins were identified that showed strong sequence matches to the human apyrase (Table 1). The greatest degree of amino acid identity is with the AK006565 gene product of Mus musculus, which shows 80% amino acid identity to the human enzyme.

The deduced amino acid of the encoded polypeptide was compared to the non-redundant protein database at the NCBI. The top seven matches are shown in Table 1 in descending order of sequence similarity.

TABLE 1
Similarity of the soluble human apyrase with members of the non-
redundant protein database.

| | | Amino | % | Accession |
|---|---|---|---|---|
| Species | Name (reference) | Acids | Identity | No. |
| Homo sapiens | Human Apyrase | 371 | — | |
| Mus musculus | Hypothetical | 440 | 80 | BAB24655 |
| | protein | | | |
| | AK006565 | | | |
| Drosophila | CG5276 | 419 | 53 | AF54638 |
| melanogaster | | | | |
| Caenorhabditis | Hypothetical | 296 | 50 | T15973 |
| elegans | protein F08C6.6 | | | |
| Cimex lectularius | Apyrase (Valen- | 364 | 45 | AAD09177 |
| | zuela et al., J. Biol. | | | |
| | Chem. | | | |
| | 273: 30583-90, | | | |
| | 1998) | | | |
| Phlebotomus | Salivary apyrase | 336 | 38 | AAG17637 |
| papatasi | | | | |
| Luzomyia | Putative apyrase | 325 | 34 | AAD33513 |
| longipalpi | (Charlab et al., | | | |
| | Proc. Natl. Acad. | | | |
| | Sci. (USA) | | | |
| | 96:15155-60, | | | |
| | 1999) | | | |
| Caenorhabditis | Hypothetical | 317 | 40 | T33887 |
| elegans | protein | | | |
| | Y39F10A.2 | | | |

APYR Nucleic Acids

The nucleic acids of the invention include those that encode an APYR polypeptide or protein. As used herein, the terms polypeptide and protein are interchangeable.

In some embodiments, an APYR nucleic acid encodes a mature APYR polypeptide. As used herein, a “mature” form of a polypeptide or protein described herein relates to the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an open reading frame described herein. A preferred mature polypeptide includes amino acids 39-371 of SEQ ID NO:2. While a preferred leader peptide includes amino acids 1-38 of SEQ ID NO:2, it should be understood by those skilled in the art that various other leader sequences may be used so long as the functioning peptide can be formed.

In some embodiments, the product “mature” form arises as a result of one or more naturally occurring processing steps that may take place within the cell in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus, a mature form arising from a precursor polypeptide or protein that has residues 1 to N, where residue 1 is the N-termninal methionine, would have residues 2 through N remaining after removal of the N-terminal methionine. Alternatively, a mature fonn arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

Among the APYR nucleic acids is the nucleic acid whose sequence is provided in SEQ ID NO:1, or a fragment thereof. Additionally, the invention includes mutant or variant nucleic acids of SEQ ID NO:1, or a fragment thereof, any of whose bases may be changed from the corresponding bases shown in SEQ ID NO:1, while still encoding a protein that maintains at least one of its APYR-like activities and physiological functions (i.e., modulating platelet aggregation). The invention further includes the complement of the nucleic acid sequence of SEQ ID NO:1, including fragments, derivatives, analogs and homologs thereof. The invention additionally includes nucleic acids or nucleic acid fragments, or complements thereto, whose structures include chemical modifications.

One aspect of the invention pertains to isolated nucleic acid molecules that encode APYR proteins or biologically active portions thereof. Also included are nucleic acid fragments sufficient for use as hybridization probes to identify APYR-encoding nucleic acids (e.g., APYR mRNA) and fragments for use as polymerase chain reaction (PCR) primers for the amplification or mutation of APYR nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated APYR nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

“Probes” refer to nucleic acid sequences of variable length, preferably at least about 10 nucleotides (nt), but can be about 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

A nucleic acid molecule of the present invention, e.g. a nucleic acid molecule having the nucleotide sequence of SEQ ID NO:1, or a complement thereof, can be isolated using standard molecular biology techniques and the sequence information provided herein. Using all or a portion of the nucleic acid sequence of SEQ ID NO:1 as a hybridization probe, APYR nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to APYR nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 25 nt, 50 nt, or 100 nt in length, preferably about 15 nt to 30 nt in length. In one embodiment, an oligonucleotide comprising a nucleic acid molecule less than 100 nt in length would further comprise at least 6 contiguous nucleotides of SEQ ID NO:1, or a complement thereof. Oligonucleotides may be chemically synthesized and may be used as probes.

In another embodiment, an isolated nucleic acid molecule of the invention comprises a nucleic acid molecule that is a complement of the nucleotide sequence shown in SEQ ID NO:1, or a portion of this nucleotide sequence. A nucleic acid molecule that is complementary to the nucleotide sequence shown in SEQ ID NO:1 is one that is sufficiently complementary to the nucleotide sequence shown in SEQ ID NO:1 that it can bind with few or no mismatches to the nucleotide sequence shown in SEQ ID NO:1, thereby forming a stable duplex.

As used herein, the term “complementary” refers to Watson-Crick or Hoogsteen base pairing between nucleotide units of a nucleic acid molecule, and the term “bind” or “binding” means the physical or cheinical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A nucleic acid molecule of the invention may include only a portion of the nucleic acid sequence of SEQ ID NO:1, e.g., a fragment that can be used as a probe or primer, or a fragment encoding a biologically active portion of APYR. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compouid but differ from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithmn of Smith and Waterman (Adv. Appl. Math., 1981, 2: 482-489, which is incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of an APYR polypeptide. Isoforms can be expressed in different tissues of the same organism as a result of, for example, alternative splicing of RNA. Alternatively, different genes can encode isoforms. In the present invention, homologous nucleotide sequences include nucleotide sequences encoding for an APYR polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human APYR protein. Homologous nucleic acid sequences include those nucleic acid sequences that encode conservative amino acid substitutions (see below) in SEQ ID NO:2, as well as a polypeptide having APYR activity. Biological activities of the APYR proteins are described below. A homologous amino acid sequence does not encode the amino acid sequence of a human APYR polypeptide.

The nucleotide sequence determined from the cloning of the human APYR gene allows for the generation of probes and primers designed for use in identifying and/or cloning APYR homologues in other cell types, e.g., from other tissues, as well as APYR homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO:1, or an anti-sense strand nucleotide sequence of SEQ ID NO:1, or of a naturally occurring mutant of SEQ ID NO:1.

Probes based on the human APYR nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an APYR protein, such as by measuring a level of an APYR-encoding nucleic acid in a sample of cells from a subject e.g., detecting APYR mRNA levels or determining whether a genomic APYR gene has been mutated or deleted.

A “polypeptide having a biologically active portion of APYR” refers to polypeptides exhibiting activity similar, but not necessarily identical to, an activity of a polypeptide of the present invention, including mature forms, as measured in a particular biological assay, with or without dose dependency. In some embodiments, the biological activity is nucleotide hydrolysis, e.g., ATP or ADP hydrolysis, or hydrolysis of both nucleotides.

A nucleic acid fragment encoding a “biologically active portion of APYR ” can be prepared by isolating a portion of SEQ ID NO:1 that encodes a polypeptide having an APYR biological activity (biological activities of the APYR proteins are described below), expressing the encoded portion of APYR protein (e.g., by recombinant expression in vitro) and assessing the activity of the encoded portion of APYR. For example, a nucleic acid fragment encoding a biologically active portion of APYR can optionally include an ATP-binding domain. In another embodiment, a nucleic acid fragment encoding a biologically active portion of APYR includes one or more regions.

APYR Variants

The invention further encompasses nucleic acid molecules that differ from the nucleotide sequences shown in SEQ ID NO:1 due to the degeneracy of the genetic code. These nucleic acids thus encode the same APYR protein as that encoded by the nucleotide sequence shown in SEQ ID NO:1, e.g., the polypeptide of SEQ ID NO:2, respectively.

In addition to the human APYR nucleotide sequence shown in SEQ ID NO:1, it will be appreciated by those skilled in the art that DNA sequence polymorphisms that lead to changes in the amino acid sequences of APYR may exist within a population (e.g., the human population). Such genetic polymorphism in the APYR gene may exist among individuals within a population due to natural allelic variation. As used herein, the terms “gene” and “recombinant gene” refer to nucleic acid molecules comprising an open reading frame encoding an APYR protein, preferably a mammalian APYR protein. Such natural allelic variations can typically result in 1-5% variance in the nucleotide sequence of the APYR gene. Any and all such nucleotide variations and resulting amino acid polymorphisms in APYR that are the result of natural allelic variation and that do not alter the functional activity of APYR are intended to be within the scope of the invention.

Moreover, nucleic acid molecules encoding APYR proteins from other species, and thus that have a nucleotide sequence that differs from the human sequence of SEQ ID NO:1 are intended to be within the scope of the invention. Nucleic acid molecules corresponding to natural allelic variants and homologues of the APYR cDNAs of the invention can be isolated based on their homology to the human APYR nucleic acids disclosed herein using the human cDNAs, or a portion thereof, as a hybridization probe according to standard hybridization techniques under stringent hybridization conditions. For example, a soluble human APYR cDNA can be isolated based on its homology to human membrane-bound APYR. Likewise, a membrane-bound human APYR cDNA can be isolated based on its homology to soluble human APYR.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:1. In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding APYR proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A nonlimiting example of stringent hybridization conditions is hybridization in a high salt buffer comprising 6× SSC, 50 mM Tris-HCl (pH 7.5), 1 nM EDTA, 0.02% PVP, 0.02% Ficoll, 0.02% BSA, and 500 mg/ml denatured salmon sperm DNA at 65° C. This hybridization is followed by one or more washes in 0.2× SSC, 0.01% BSA at 50° C. An isolated nucleic acid molecule of the invention that hybridizes under stringent conditions to the sequence of SEQ ID NO:1 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:1, or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0. 1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO:1, or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative Mutations

In addition to naturally-occurring allelic variants of the APYR sequence that may exist in the population, the skilled artisan will further appreciate that changes can be introduced by mutation into the nucleotide sequence of SEQ ID NO:1, thereby leading to changes in the amino acid sequence of the encoded APYR protein, without altering the functional ability of the APYR protein. For example, nucleotide substitutions leading to amino acid substitutions at “non-essential” amino acid residues can be made in the sequence of SEQ ID NO:1. A “non-essential” amino acid residue is a residue that can be altered from the wild-type sequence of APYR without altering the biological activity, whereas an “essential” amino acid residue is required for biological activity. For example, amino acid residues that are conserved among the APYR proteins of the present invention, are predicted to be particularly unamenable to alteration.

Another aspect of the invention pertains to nucleic acid molecules encoding APYR proteins that contain changes in amino acid residues that are not essential for activity. Such APYR proteins differ in amino acid sequence from SEQ ID NO:2, yet retain biological activity. In one embodiment, the isolated nucleic acid molecule comprises a nucleotide sequence encoding a protein, wherein the protein comprises an amino acid sequence at least about 75% homologous to the amino acid sequence of SEQ ID NO:2. Preferably, the protein encoded by the nucleic acid is at least about 80% homologous to SEQ ID NO:2, more preferably at least about 90%, 95%, 98%, and most preferably at least about 99% homologous to SEQ ID NO:2.

An isolated nucleic acid molecule encoding an APYR protein homologous to the protein of SEQ ID NO:2 can be created by introducing one or more nucleotide substitutions, additions or deletions into the nucleotide sequence of SEQ ID NO:1, such that one or more amino acid substitutions, additions or deletions are introduced into the encoded protein.

Mutations can be introduced into the nucleotide sequence of SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valnie, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in APYR is replaced with another amino acid residue from the same side chain family. Alternatively, in another embodiment, mutations can be introduced randomly along all or part of an APYR coding sequence, such as by saturation mutagenesis, and the resultant mutants can be screened for APYR biological activity to identify mutants that retain activity. Following mutagenesis of SEQ ED NO:1 the encoded protein can be expressed by any recombinant technology known in the art and the activity of the protein can be determined.

In one embodiment, a mutant APYR protein can be assayed for (1) the ability to form protein:protein interactions with other APYR proteins, other cell-surface proteins, or biologically active portions thereof, (2) complex formation between a mutant APYR protein and an APYR receptor; (3) the ability of a mutant APYR protein to bind to an intracellular target protein or biologically active portion thereof; (e.g., avidin proteins); (4) the ability to bind APYR protein; or (5) the ability to specifically bind an anti-APYR protein antibody.

Antisense APYR Nucleic Acids

Another aspect of the invention pertains to isolated antisense nucleic acid molecules that are hybridizable to or complementary to the nucleic acid molecule comprising the nucleotide sequence of SEQ ID NO:1, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire APYR coding strand, or to only a portion thereof. Nucleic acid molecules encoding fragments, homologs, derivatives and analogs of an APYR protein of SEQ ID NO:2, or antisense nucleic acids complementary to an APYR nucleic acid sequence of SEQ ID NO:1 are additionally provided.

In one embodiment, an antisense nucleic acid molecule is antisense to a “coding region” of the coding strand of a nucleotide sequence encoding APYR . The term “coding region” refers to the region of the nucleotide sequence comprising codons which are translated into amino acid residues (e.g., the protein coding region of human APYR corresponds to SEQ ID NO:2). In another embodiment, the antisense nucleic acid molecule is antisense to a “noncoding region” of the coding strand of a nucleotide sequence encoding APYR. The term “noncoding region” refers to 5′ and 3′ sequences that flank the coding region that are not translated into amino acids (i.e., also referred to as 5′ and 3′ untranslated regions).

Given the coding strand sequences encoding APYR disclosed herein (e.g., SEQ ID NO:1), antisense nucleic acids of the invention can be designed according to the rules of Watson and Crick or Hoogsteen base pairing. The antisense nucleic acid molecule can be complementary to the entire coding region of APYR mRNA, but more preferably is an oligonucleotide that is antisense to only a portion of the coding or noncoding region of APYR mRNA. For example, the antisense oligonucleotide can be complementary to the region surrounding the translation start site of APYR mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcyto sine, 5-methylcyto sine, N6-adenine, 7-methylguanine, 5-methylaminomethyluracil, 5-methoxyaminomethyl-2-thiouracil, beta-D-mamiosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an anitisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an APYR protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systenmic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

Nucleic Acid Arrays

The present invention further provides nucleic acid detection kits, such as arrays or microarrays of nucleic acid molecules that are based on the sequence information provided in FIG. 1 (SEQ ID NOS:1). As used herein “Arrays” or “Microarrays” refers to an array of distinct polynucleotides or oligonucleotides synthesized on a substrate, such as paper, nylon or other type of membrane, filter, chip, glass slide, or any other suitable solid support. In one embodiment, the microarray is prepared and used according to the methods described in U.S. Pat. No. 5,837,832, Chee et al., In other embodiments, such arrays are produced by the methods described by Brown et al., U.S. Pat. No. 5,807,522.

The microarray or detection kit is preferably composed of a large number of unique, single-stranded nucleic acid sequences, usually either synthetic antisense oligonucleotides or fragments of cDNAs, fixed to a solid support. The oligonucleotides are preferably about 4-60 nucleotides in length, more preferably 5-30 nucleotides in length, and most preferably about 10-25 nucleotides in length. For a certain type of microarray or detection kit, it may be preferable to use oligonucleotides that are only 4-18 nucleotides in length.

Using such arrays, the present invention provides methods to identify the expression of the APYR proteins/peptides of the present invention. Such assays will typically involve arrays comprising many genes, at least one of which is a gene of the present invention and or alleles of the APYR gene of the present invention.

Nucleic acid expression assays are useful for drug screening to identify compounds that modulate APYR nucleic acid expression. The invention thus provides a method for identifying a compound that can be used to treat a disorder associated with nucleic acid expression of the APYR gene, particularly biological and pathological processes that are mediated by the APYR in cells and tissues that express it.

The assay for APYR nucleic acid expression can involve direct assay of nucleic acid levels, such as mRNA levels. In this embodiment the regulatory regions of these genes can be operably linked to a reporter gene such as luciferase.

Thus, modulators of APYR gene expression can be identified in a method wherein a cell is contacted with a candidate compound and the expression of mRNA determined. The level of expression of APYR mRNA in the presence of the candidate compound is compared to the level of expression of APYR mRNA in the absence of the candidate compound. The candidate compound can then be identified as a modulator of nucleic acid expression based on this comparison and be used, for example to treat a disorder characterized by aberrant nucleic acid expression.

The invention further provides methods of treatment, with the nucleic acid as a target, using a compound identified through drug screening as a gene modulator to modulate APYR nucleic acid expression in cells and tissues that express the APYR. Modulation includes both up-regulation (i.e. activation or agonization) or down-regulation (suppression or antagonization) or nucleic acid expression.

Alternatively, a modulator for APYR nucleic acid expression can be a small molecule or drug identified using the screening assays described herein as long as the drug or small molecule inhibits the APYR nucleic acid expression in the cells and tissues that express the protein.

APYR Polypeptides

The present invention provides isolated peptide and protein molecules that consist of, consist essentially of, or comprise the amino acid sequences of the APYR peptides disclosed in the FIG. 1, (encoded by the nucleic acid molecule shown in FIG. 1), as well as all obvious variants of these peptides that are within the art to make and use. Some of these variants are described in detail below.

An APYR polypeptide of the invention includes the APYR-like protein whose sequence is provided in SEQ ID NO:2. The invention also includes a mutant or variant protein any of whose residues may be changed from the corresponding residue shown in SEQ ID NO:2 while still encoding a protein that maintains its APYR-like activities and physiological functions, or a functional fragment thereof. In some embodiments, up to 20% or more of the residues may be so changed in the mutant or variant protein. In some embodiments, the APYR polypeptide according to the invention is a mature polypeptide.

In general, an APR-like variant that preserves APYR-like function (such as ATP or ADP hydrolysis) includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated APYR proteins, and biologically active portions thereof, or derivatives, fragments, analogs or homologs thereof. Also provided are polypeptide fragments suitable for use as immunogens to raise anti-APYR antibodies. In one embodiment, native APYR proteins can be isolated from cells or tissue sources by an appropriate purification scheme using standard protein purification techniques. In another embodiment, APYR proteins are produced by recombinant DNA techniques. Alternative to recombinant expression, an APYR protein or polypeptide can be synthesized chemically using standard peptide synthesis techniques.

“APYR protein” or “APYR polypeptide” refer to a protein or polypeptide encoded by the APYR locus, variants or fragments thereof. The term “polypeptide” refers to a polymer of amino acids and its equivalent and does not refer to a specific length of the product; thus, peptides, oligopeptides and proteins are included within the definition of a polypeptide. This term also does not refer to, or exclude modifications of the polypeptide, for example, glycosylations, acetylations, phosphorylations, and the like. Included within the definition are, for example, polypeptides containing one or more analogs of an amino acid (including, for example, unnatural amino acids, etc.), polypeptides with substituted linkages as well as other modifications known in the art, both naturally and non-naturally occurring.

The term “polypeptide” is used in its broadest sense, i.e., any polymer of amino acids (dipeptide or greater) linked through peptide bonds. Thus, the term “polypeptide” includes proteins, oligopeptides, protein fragments, analogs, muteins, fusion proteins and the like. “Native” proteins or polypeptides refer to proteins or polypeptides recovered from a source occurring in nature.

“Protein modifications or fragments” are provided by the present invention for APYR polypeptides or fragments thereof which are substantially homologous to primary structural sequence but which include, e.g., in vivo or in vitro chemical and biochemical modifications or which incorporate unusual amino acids. Such modifications include, for example, acetylation, carboxylation, phosphorylation, glycosylation, ubiquitination, labeling, e.g., with radionuclides, and various enzymatic modifications, as will be readily appreciated by those well skilled in the art. A variety of methods for labeling polypeptides and of substituents or labels useful for such purposes are well known in the art, and include radioactive isotopes such as ³²P, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods of labeling polypeptides are well known in the art.

Besides substantially full-length polypeptides, the present invention provides for biologically active fragments of the polypeptides. Significant biological activities include ligand-binding, immunological activity and other biological activities characteristic of APYR polypeptides. Immunological activities include both immunogenic function in a target immune system, as well as sharing of immunological epitopes for binding, serving as either a competitor or substitute antigen for an epitope of the APYR protein. As used herein, “epitope” refers to an antigenic determinant of a polypeptide. An epitope could comprise three amino acids in a spatial conformation that is unique to the epitope. Generally, an epitope consists of at least five such amino acids, and more usually consists of at least 6-10 such amino acids. Methods of determining the spatial conformation of such amino acids are known in the art.

The present invention also provides for fusion polypeptides, comprising APYR polypeptides and fragments. Homologous polypeptides may be fusions between two or more APYR polypeptide sequences or between the sequences of APYR and a related protein. Likewise, heterologous fusions may be constructed which would exhibit a combination of properties or activities of the derivative proteins. For example, ligand-binding or other domains may be “swapped” between different new fusion polypeptides or fragments. Such homologous or heterologous fusion polypeptides may display, for example, altered strength or specificity of binding. Fusion partners include immunoglobulins, bacterial beta-galactosidase, trpE, protein A, beta-lactamase, alpha amylase, alcohol dehydrogenase and yeast alpha mating factor. Fusion proteins will typically be made by either recombinant nucleic acid methods or may be chemically synthesized.

A “purified” protein or biologically active portion thereof is substantially free of cellular material or other contaminating proteins from the cell or tissue source from which the APYR protein is derived, or substantially free from chemical precursors or other chemicals when chemically synthesized. The language “substantially free of cellular material” includes preparations of APYR protein in which the protein is separated from cellular components of the cells from which it is isolated or recombinantly produced. In one embodiment, the language “substantially free of cellular material” includes preparations of APYR protein having less than about 30% (by dry weight) of non-APYR protein (also referred to herein as a “contaminating protein”), more preferably less than about 20% of non-APYR protein, still more preferably less than about 10% of non-APYR protein, and most preferably less than about 5% non-APYR protein. When the APYR protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of APYR protein in which the protein is separated from chemical precursors or other chemicals that are involved in the synthesis of the protein. In one embodiment, the language “substantially free of chemical precursors or other chemicals” includes preparations of APYR protein having less than about 30% (by dry weight) of chemical precursors or non-APYR chemicals, more preferably less than about 20% chemical precursors or non-APYR chemicals, still more preferably less than about 10% chemical precursors or non-APYR chemicals, and most preferably less than about 5% chemical precursors or non-APYR chemicals.

Biologically active portions of an APYR protein include peptides comprising amino acid sequences sufficiently homologous to or derived from the amino acid sequence of the APYR protein, e.g., the amino acid sequence shown in SEQ ID NO:2 that include fewer amino acids than the full length APYR proteins, and exhibit at least one activity of an APYR protein. Typically, biologically active portions comprise a domain or motif with at least one activity of the APYR protein. A biologically active portion of an APYR protein can be a polypeptide that is, for example, 10, 25, 50, 100 or more amino acids in length.

A biologically active portion of an APYR protein of the present invention may contain at least one of the above-identified domains conserved between the APYR proteins. Moreover, other biologically active portions, in which other regions of the protein are deleted, can be prepared by recombinant techniques and evaluated for one or more of the functional activities of a native APYR protein.

In an embodiment, the APYR protein has an amino acid sequence shown in SEQ ID NO:2. In other embodiments, the APYR protein is substantially homologous to SEQ ID NO:2 and retains the functional activity of the protein of SEQ ID NO:2, yet differs in amino acid sequence due to natural allelic variation or mutagenesis, as described in detail below. Accordingly, in another embodiment, the APYR protein is a protein that comprises an amino acid sequence at least about 45% homologous to the amino acid sequence of SEQ ID NO:2 and retains the functional activity of the APYR proteins of SEQ ID NO:2.

The isolated APYR peptide can be purified from cells that naturally express it, purified from cells that have been altered to express it (recombinant), or synthesized using known protein synthesis methods. For example, a nucleic acid molecule encoding the APYR peptide is cloned into an expression vector, the expression vector introduced into a host cell and the protein expressed in the host cell. The protein can then be isolated from the cells by an appropriate purification scheme using standard protein purification techniques. Accordingly, the present invention provides proteins that consist of the amino acid sequences provided in SEQ ID NO:2, for example, proteins encoded by the transcript/cDNA nucleic acid sequences shown in FIG. 1 (SEQ ID NO:1).

The present invention further provides proteins that consist essentially of the amino acid sequences provided in FIG. 1 (SEQ ID NO:2), for example, proteins encoded by the transcript/cDNA nucleic acid sequences shown in FIG. 1 (SEQ ID NO:1). A protein consists essentially of an amino acid sequence when such an amino acid sequence is present with only a few additional amino acid residues, for example from about 1 to about 100 or so additional residues, typically from 1 to about 20 additional residues in the final protein.

The present invention further provides proteins that comprise the amino acid sequences provided in FIG. 1 (SEQ ID NO:2), for example, proteins encoded by the transcript/cDNA nucleic acid sequences shown in FIG. 1 (SEQ ID NO:1). A protein comprises an amino acid sequence when the amino acid sequence is at least part of the final amino acid sequence of the protein. In such a fashion, the protein can be only the peptide or have additional amino acid molecules, such as amino acid residues (contiguous encoded sequence) that are naturally associated with it or heterologous amino acid residues/peptide sequences. Such a protein can have a few additional amino acid residues or can comprise several hundred or more additional amino acids. The preferred classes of proteins that are comprised of the APYR peptides of the present invention are the naturally occurring mature proteins.

Determining Homology Between Two or More Sequences

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:1.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determing the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

As mentioned above, the present invention also provides and enables obvious variants of the amino acid sequence of the proteins of the present invention, such as naturally occurring mature forms of the peptide, allelic/sequence variants of the peptides, non-naturally occurring recombinantly derived variants of the peptides, and orthologs and paralogs of the peptides. Such variants can readily be generated using art-known techniques in the fields of recombinant nucleic acid technology and protein biochemistry. It is understood, however, that variants exclude any amino acid sequences disclosed prior to the invention.

Such variants can readily be identified/made using molecular techniques and the sequence information disclosed herein. Further, such variants can readily be distinguished from other peptides based on sequence and/or structural homology to the APYR peptides of the present invention. The degree of homology/identity present will be based primarily on whether the peptide is a functional variant or non-functional variant, the amount of divergence present in the paralog family and the evolutionary distance between the orthologs.

To determine the percent identity of two amino acid sequences or two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, at least 30%, 40%, 50%, 60%, 70%, 80%, or 90% or more of the length of a reference sequence is aligned for comparison purposes. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The nucleic acid and protein sequences of the present invention can further be used as a “query sequence” to perform a search against sequence databases to, for example, identify other family members or related sequences. Full-length pre-processed forms, as well as mature processed forms, of proteins that comprise one of the peptides of the present invention can readily be identified as having complete sequence identity to one of the APYR peptides of the present invention as well as being encoded by the same genetic locus as the APYR peptide provided herein.

Paralogs of an APYR peptide can readily be identified as having some degree of significant sequence homology/identity to at least a portion of the APYR peptide, as being encoded by a gene from humans, and as having similar activity or function. Two proteins will typically be considered paralogs when the amino acid sequences are typically at least about 60% or greater, and more typically at least about 70% or greater homology through a given region or domain. Such paralogs will be encoded by a nucleic acid sequence that will hybridize to an APYR peptide encoding nucleic acid molecule under moderate to stringent conditions as more fully described below.

Orthologs of an APYR peptide can readily be identified as having some degree of significant sequence homology/identity to at least a portion of the APYR peptide as well as being encoded by a gene from another organism. Preferred orthologs will be isolated from mammals, preferably primates, for the development of human therapeutic targets and agents. Such orthologs will be encoded by a nucleic acid sequence that will hybridize to an APYR peptide encoding nucleic acid molecule under moderate to stringent conditions, as more fully described below, depending on the degree of relatedness of the two organisms yielding the proteins.

Non-naturally occurring variants of the APYR peptides of the present invention can readily be generated using recombinant techniques. Such variants include, but are not limited to deletions, additions and substitutions in the amino acid sequence of the APYR peptide. For example, one class of substitutions is conserved amino acid substitution. Such substitutions are those that substitute a given amino acid in an APYR peptide by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr.

Variant APYR peptides can be fully functional or can lack function in one or more activities, e.g. ability to bind substrate, ability to hydrolyze substrate, etc. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

The present invention further provides fragments of the APYR peptides, in addition to proteins and peptides that comprise and consist of such fragments, particularly those comprising the residues identified in FIG. 2. The fragments to which the invention pertains, however, are not to be construed as encompassing fragments that may be disclosed publicly prior to the present invention.

Polypeptides often contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in APYR peptides are described in basic texts, detailed monographs, and the research literature, and they are well known to those of skill in the art (some of these features are identified in FIG. 2).

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, are well known in the art.

Accordingly, the APYR peptides of the present invention also encompass derivatives or analogs in which a substituted amino acid residue is not one encoded by the genetic code, in which a substituent group is included, in which the mature APYR peptide is fused with another compound, such as a compound to increase the half-life of the APYR peptide (for example, polyethylene glycol), or in which the additional amino acids are fused to the mature APYR peptide, such as a leader or secretory sequence or a sequence for purification of the mature APYR peptide or a pro-protein sequence.

Chimeric and Fusion Proteins

The invention also provides APYR chimeric or fusion proteins. As used herein, an APYR “chimeric protein” or “fusion protein” comprises an APYR polypeptide operatively linked to a non-APYR polypeptide. An “APYR polypeptide” refers to a polypeptide having an amino acid sequence corresponding to APYR, whereas a “non-APYR polypeptide” refers to a polypeptide having an amino acid sequence corresponding to a protein that is not substantially homologous to the APYR protein, e.g., a protein that is different from the APYR protein and that is derived from the same or a different organism. Within an APYR fusion protein the APYR polypeptide can correspond to all or a portion of an APYR protein. In one embodiment, an APYR fusion protein comprises at least one biologically active portion of an APYR protein. In another embodiment, an APYR fusion protein comprises at least two biologically active portions of an APYR protein. Within the fusion protein, the term “operatively linked” is intended to indicate that the APYR polypeptide and the non-APYR polypeptide are fused in-frame to each other. The non-APYR polypeptide can be fused to the N-terminus or C-terminus of the APYR polypeptide.

For example, in one embodiment an APYR fusion protein comprises an APYR polypeptide operably linked to either an extracellular domain of a second protein, i.e., non-APYR protein, or to the transmembrane and intracellular domain of a second protein, i.e., non-APYR protein. Such fusion proteins can be further utilized in screening assays for compounds that modulate APYR activity (such assays are described in detail below).

In another embodiment, the fusion protein is a GST-APYR fusion protein in which the APYR sequences are fused to the C-terminus of the GST (i.e., glutathione S-transferase) sequences. Such fusion proteins can facilitate the purification of recombinant APYR.

In another embodiment, the fusion protein is an APYR-immunoglobulin fusion protein in which the APYR sequences comprising one or more domains are fused to sequences derived from a member of the immunoglobulin protein family.

An APYR chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended ternini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). AN APYR-encoding nucleic acid can be cloned into such an expression vector such that the fusion moiety is linked in-frame to the APYR protein.

Polypeptide Libraries

In addition, libraries of fragments of the APYR protein coding sequence can be used to generate a variegated population of APYR fragments for screening and subsequent selection of variants of an APYR protein. In one embodiment, a library of coding sequence fragments can be generated by treating a double stranded PCR fragment of an APYR coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the APYR protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of APYR proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify APYR variants (Arkin and Yourvan (1992) PNAS 89:7811-7815; Delgrave et al. (1993) Protein Engineering 6:327-331).

The proteins of the present invention can be used in substantial and specific assays related to the functional information provided in the Figures; to raise antibodies or to elicit another immune response; as a reagent (including the labeled reagent) in assays designed to quantitatively determine levels of the protein (or its binding partner or ligand) in biological fluids; and as markers for tissues in which the corresponding protein is preferentially expressed (either constitutively or at a particular stage of tissue differentiation or development or in a disease state). Where the protein binds or potentially binds to another protein or ligand (such as, for example, in an APYR-effector protein interaction or APYR-ligand interaction), the protein can be used to identify the binding partner/ligand so as to develop a system to identify inhibitors of the binding interaction. Any or all of these uses are capable of being developed into reagent grade or kit format for commercialization as commercial products.

The peptides are also useful for treating a disorder characterized by an absence of, inappropriate, or unwanted expression of the protein. Accordingly, methods for treatment include the use of the APYR protein or fragments.

APYR Antibodies

Also included in the invention are antibodies to APYR proteins, or fragments of APYR proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library.

An isolated APYR-related protein of the invention may be used as an antigen, or a portion or fragment thereof, and additionally can be used as an immunogen to generate antibodies that immunospecifically bind the antigen, using standard techniques for polyclonal and monoclonal antibody preparation. The full-length protein or mature portion (e.g., amino acids 39-371 of SEQ ID NO:2) can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NO:2, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of APYR-related protein that is located on the surface of the protein, e.g., a hydrophilic region. A hydrophobicity analysis of the human APYR-related protein sequence will indicate which regions of an APYR-related protein are particularly hydrophilic and, therefore, are, likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety.. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an inmmunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., and Kohler and Milstein, Nature, 256:495 (1975).

APYR Recombinant Expression Vectors and Host Cells

Another aspect of the invention pertains to vectors, preferably expression vectors, containing a nucleic acid encoding an APYR protein, or derivatives, fragments, analogs or homologs thereof. As used herein, the term “vector” refers to a nucleic acid molecule capable of transporting another nucleic acid to which it has been linked. With this aspect of the invention, the vector includes a plasmid, single or double stranded phage, a single or double stranded RNA or DNA viral vector, or artificial chromosome, such as a BAC, PAC, YAC, OR MAC. The invention provides vectors for the maintenance (cloning vectors) or vectors for expression (expression vectors) of the nucleic acid molecules. The vectors can function in prokaryotic or eukaryotic cells or in both (shuttle vectors).

One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g. polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., APYR proteins, mutant forms of APYR proteins, fusion proteins, etc.).

The regulatory sequences to which the nucleic acid molecules described herein can be operably linked include promoters for directing mRNA transcription. These include, but are not limited to, the left promoter from bacteriophage, the lac, TRP, and TAC promoters from E. coli, the early and late promoters from SV40, the CMV immediate early promoter, the adenovirus early and late promoters, and retrovirus long-temiinal repeats. In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

The recombinant expression vectors of the invention can be designed for expression of APR proteins in prokaryotic or eukaryotic cells. For example, APYR proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated inl vitro, for example using T7 promoter regulatory sequences and T7 polymerase.

In another embodiment, the APYR expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gense 54: 113-123), pYES2 (Invitrogen Corporation, San Diego, Calif.), and picZ (InVitrogen Corp, San Diego, Calif.).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to APYR mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, APYR protein can be expressed in bacterial cells such as E. Coli, insect cells, yeast or mammalian cells (such as human, Chinese hamster ovary cells (CHO) or COS cells). Other suitable host cells are known to those skilled in the art. Vector DNA can be introduced into prokaryotic or eukaryotic cells via conventional transformation or transfection techniques.

A host cell of the invention, such as a prokaryotic or eukaryotic host cell in culture, can be used to produce (i.e., express) APYR protein. Accordingly, the invention further provides methods for producing APYR protein using the host cells of the invention. In one embodiment, the method comprises culturing the host cell of invention (into which a recombinant expression vector encoding APYR protein has been introduced) in a suitable medium such that APYR protein is produced. In another embodiment, the method further comprises isolating APYR protein from the medium or the host cell.

Transgenic APYR Animals

The host cells of the invention can also be used to produce non-human transgenic animals. For example, in one embodiment, a host cell of the invention is a fertilized oocyte or an embryonic stem cell into which APYR protein-coding sequences have been introduced. Such host cells can then be used to create non-human transgenic animals in which exogenous APYR sequences have been introduced into their genome or homologous recombinant animals in which endogenous APYR sequences have been altered. Such animals are useful for studying the function and/or activity of APYR protein and for identifying and/or evaluating modulators of APYR protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-hunman animal, preferably a mammal, more preferably a mouse, in which an endogenous APYR gene has been altered by homologous recombination between the endogenous gene and an exogenous DNA molecule introduced into a cell of the animal, e.g., an embryonic cell of the animal, prior to development of the animal.

Introducing APYR-encoding nucleic acid into the male pronuclei of a fertilized oocyte (e.g., by microinjection, retroviral infection) and allowing the oocyte to develop in a pseudopregnant female foster animal can create a transgenic animal of the invention. Sequences including SEQ ID NO:1 can be introduced as a transgene into the genome of a non-human animal. Alternatively, a non-human homologue of the human APYR gene, such as a mouse APYR gene, can be isolated based on hybridization to the human APYR cDNA (described further supra) and used as a transgene. Intronic sequences and polyadenylation signals can also be included in the transgene to increase the efficiency of expression of the transgene. A tissue-specific regulatory sequence(s) can be operably linked to the APYR transgene to direct expression of APYR protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y.. Similar methods are used for production of other transgenic animals. A transgenic founder animal can be identified based upon the presence of the APYR transgene in its genome and/or expression of APYR mRNA in tissues or cells of the animals. A transgenic founder animal can then be used to breed additional animals carrying the transgene. Moreover, transgenic animals carrying a transgene-encoding APYR protein can further be bred to other transgenic animals carrying other transgenes.

Pharmaceutical Compositions

The APYR nucleic acid molecules, APYR proteins, and anti-APYR antibodies (also referred to herein as “active compounds”) of the invention, and derivatives, fragments, analogs and homologs thereof, can be incorporated into pharmaceutical compositions suitable for administration. Such compositions typically comprise an APYR nucleic acid molecule, an APYR protein, or an APYR antibody, and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Various antibacterial and antifungal agents, for example, parabens, chlorobutanol, phenol, ascorbic acid, thimerosal, and the like can achieve prevention of the action of microorganisms. In many cases, it will be preferable to include isotonic agents, for example, sugars, polyalcohols such as manitol, sorbitol, sodium chloride in the composition. Including in the composition an agent which delays absorption, for example, aluminum monostearate and gelatin, can bring about prolonged absorption of the injectable compositions.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdennal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The pharmaceutical compositions can be included in a container, pack, or dispenser together with instructions for administration.

Screening and Detection Methods

The isolated nucleic acid molecules of the invention can be used to express APYR protein (e.g., via a recombinant expression vector in a host cell in gene therapy applications), to detect APYR mRNA (e.g., in a biological sample) or a genetic lesion in an APYR gene, and to modulate APYR activity, as described further, below. In addition, the APYR proteins can be used to screen drugs or compounds that modulate the APYR protein activity or expression as well as to treat disorders characterized by insufficient or excessive production of APYR protein or production of APYR protein forms that have decreased or aberrant activity compared to APYR wild-type protein. In addition, the anti-APYR antibodies of the invention can be used to detect and isolate APYR proteins and modulate APYR activity. For example, APYR activity includes T-cell or NK cell growth and differentiation, antibody production, and tumor growth.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to APYR proteins or have a stimulatory or inhibitory effect on, e.g., APYR protein expression or APYR protein activity. The invention also includes compounds identified in the screening assays described herein.

The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of APYR protein, or a biologically active portion thereof, on the cell surface is contacted with a test compound and the ability of the test compound to bind to an APYR protein determined. The cell, for example, can be of mammalian origin or a yeast cell. Determining the ability of the test compound to bind to the APYR protein can be accomplished, for example, by coupling the test compound with a radioisotope or enzymatic label such that binding of the test compound to the APYR protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of APYR protein, or a biologically-active portion thereof, on the cell surface with a known compound which binds APYR to form an assay mixture, contacting the assay mixture with a test compound, and determining the ability of the test compound to interact with an APYR protein, wherein determining the ability of the test compound to interact with an APYR protein comprises determining the ability of the test compound to preferentially bind to APYR protein or a biologically-active portion thereof as compared to the known compound.

In one embodiment, the assay is a cell-free assay that includes contacting an APYR protein or biologically-active portion thereof with a test compound and determining the ability of the test compound to bind to the APYR protein or biologically-active portion thereof. Binding of the test compound to the APYR protein can be determined either directly or indirectly. In one such embodiment, the assay comprises contacting the APYR protein or biologically-active portion thereof with a known compound which binds APYR to form an assay mixture, contacting the assay mixture with a test compound, and determining the ability of the test compound to interact with an APYR protein, wherein determining the ability of the test compound to interact with an APYR protein comprises determining the ability of the test compound to preferentially bind to APYR or biologically-active portion thereof as compared to the known compound.

In still another embodiment, an assay is a cell-free assay comprising contacting APYR protein or biologically-active portion thereof with a test compound and determining the ability of the test compound to modulate (e.g. stimulate or inhibit) the activity of the APYR protein or biologically-active portion thereof. Determining the ability of the test compound to modulate the activity of APYR can be accomplished, for example, by determining the ability of the APYR protein to bind to an APYR target molecule by one of the methods described above for determining direct binding.

The invention further pertains to novel agents identified by the aforementioned screening assays and uses thereof for treatments as described herein.

The invention will be further illustrated in the following non-limiting examples.

### EXAMPLE 1

### Expression of AYPR Polypeptides in COS-1 Cells and Demonstration of Nucleotide Hydrolase Activity

Because the encoded polypeptide sequence showed strong amino acid similarity to a previously cloned bed bug apyrase, the human cDNA was expressed in mammalian COS-1 cells and the protein product analyzed for apyrase activity.

For transient transfection, COS-1 cells were grown in complete DMEM (Dulbecco's modified Eagle medium with 10% calf serum and 100 Units/ml penicillin G, 100 μg/ml streptomycin sulfate, and 0.25 μg/ml amphotericin B as Fungizone) at 37° C. in 5% CO₂. Cells were grown in this manner and passaged every 3-4 days. Cells were seeded the day prior to transfection in 100 mm plates so as to be approximately 75% confluent when transfected 20-24 hours later. Briefly, the cells were incubated for 5 hours in DMEM (serum and antibiotic/antimycotic free) containing Lipofectamine Plus reagents and either 4 μg human apyrase cDNA in pcDNA3 or 4 μg pcDNA3 vector alone (mock transfection). At the end of the 5 hour transfection, complete DMEM was added to the transfected cells. For harvesting the cell media containing soluble apyrase, the COS-1 cells were transferred to incomplete DMEM media 24 hours after transfection (48 hours before harvesting the media). Apyrase transfected and mock-transfected cell media was collected and centrifuged at 48,000 rpm for 1 hour to remove particulate material. The clarified media, approximately 30 ml, was concentrated to 1.5 mil using a Centriprep-30 MWCO ultrafiltration device, as recommended by the manufacturer (Amicon). The concentrated cell media from both mock-transfected and apyrase transfected was then used for subsequent analyses.

Conditioned cell media from human apyrase transfected COS cells were concentrated and subjected to size exclusion chromatography on a Sephacryl S-200 column. Concentrated conditioned cell media from apyrase transfected or mock-transfected cells was loaded onto a Sephacryl S-200 column (27 cm long) with a bed volume of 46 ml. The column was previously equilibrated in 50 mM Tris-HCl buffer (pH 8.5) containing 100 mM NaCl, and 5 mM CaCl₂. The column was eluted at a flow rate of 0.5 ml/min and 1 ml fractions were collected and assayed for nucleotidase activity as described above. To calibrate the column, gel-filtration standards (Bio-Rad; Bovine serum albumin, chicken ovalbumin, Equine myoglobin, Vitamin B-12) were run and the fractions analyzed by SDS-PAGE and protein assay to determine elution volumes of the component standard proteins.

Fractions were collected and assayed for calcium-dependent ADPase activity. Nucleotidase activities were determined by measuring the amount of inorganic phosphate released from nucleotide substrates at 37° C. using a modification (Kirley, J. Biol. Chem. 263: 12682-89, 1988) of the technique of Fiske et al., J. Biol. Chem. 66, 375-400 1925. Briefly, apyrase activity was measured in both mock-transfected and apyrase transfected COS cell conditioned media by incubation of concentrated media in 50 mM Tris-HCl buffer (pH 8.5) containing 100 mM NaCl, and either 5 mM CaCl₂ or 5 mM MgCl₂, depending on the cation under examination. For inhibitor testing, sodium azide was added to this reaction mixture at final concentrations of 0.1, 1, 5, 10, and 20 mM. The enzyme assay was initiated by the addition of the respective nucleotide to a final concentration of 2.5 mM and the inorganic phosphate released was determined colormetrically.

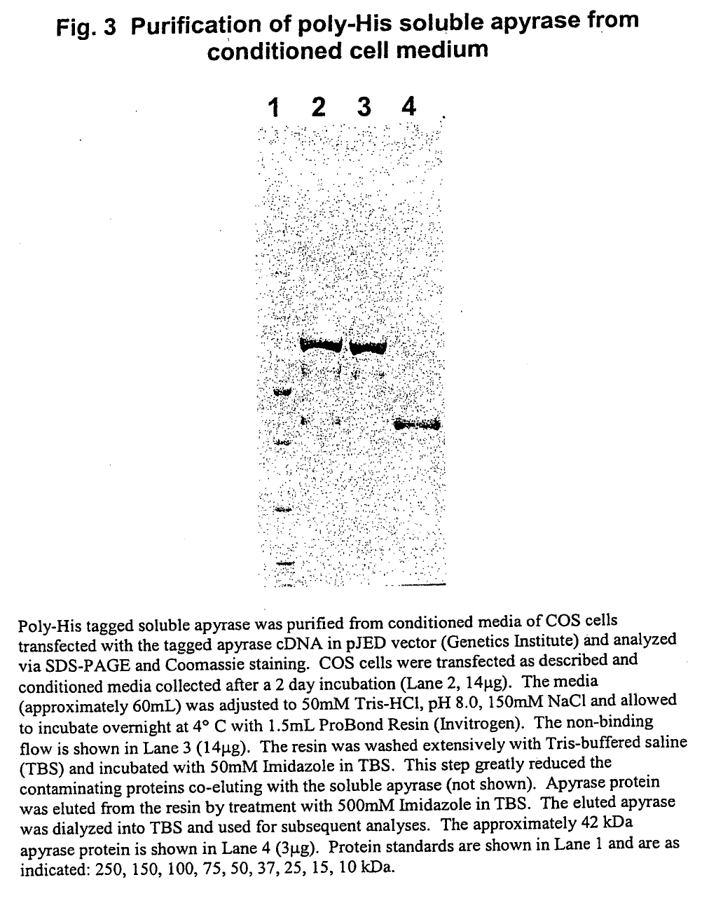

The results are shown in FIG. 3. The human soluble apyrase activity (open box) eluted with a calculated molecular mass of 34 kDa. Column standards (closed circles) were 66, 44, 17, and 1.35 kDa. Conditioned cell media was concentrated and assayed for cation-dependent ATPase and ADPase activity. The assay revealed a calcium-dependent ADPase activity present in the media of COS-1 cells transfected with the human apyrase cDNA (FIG. 2). A lessor amount of ATPase activity was also detected, but only in the presence of calcium. The human apyrase did not hydrolyze ATP or ADP to any significant extent in the presence magnesium (see FIG. 2). No apyrase activity was detected in the media of those cells mock-transfected with pcDNA3 vector alone.

Sodium azide in the range of 5-20 mM has traditionally been used as an inhibitor of extracellular E-type apyrase activity (Plesner, Int. Rev. Cyt. 158:141-214, 1995; Cote et al., Biochimica et Biophysica Acta 1160:246-50, 1992; Caldwell et al., Arch. Biochem. Biophys. 362:46-58, 1999; Caldwell et al., Arch. Biochem. Biophys. 362:46-58, 1999). In particular, azide has been shown to have a much more dramatic effect inhibiting the ADPase activity of apyrases, as compared to the ATPase activity (Smith et al., Biochim. Biophys. Acta 1386:65-78, 1998; Knowles et al., in Ecto-ATPases and Related Ectonucleotidases (Vanduffel, L., and Lemmiens, R., Eds.) pp. 39-52, Shaker Publishing B. V., Maastricht, 2000. To determine if azide had the same inhibitory effects on this soluble apyrase, apyrase transfected conditioned cell media was incubated in the presence of various concentrations of sodium azide (0.1-20 mM) and analyzed for calcium ADPase activity. Azide had no inhibitory effects on the enzymatic activity of the soluble apyrase under these conditions, with all of the activity remaining after incubation with these concentrations of azide.

To determine the molecular mass of the native human apyrase, size-exclusion chromatography was performed on the expressed human apyrase. Conditioned cell media was concentrated and applied to a Sephacryl S-200 size-exclusion column. Analyzing the eluted fraction for calcium-dependent ADPase activity revealed that the expressed enzyme had a native molecular mass of approximately 34 kDa, similar to the 37 kDa predicted for the processed form of the human apyrase (FIG. 3).

### EXAMPLE 2

### APYR Nucleic Acids are Expressed in Multiple Tissues

To determine the tissue-specific mRNA distribution of the soluble human apyrase, a human multiple tissue expression array was analyzed by hybridization with the full-length human apyrase cDNA. Tissue mRNA analysis was performed using a Multiple Tissue RNA Master Blot (Clontech). The human apyrase probe was generated by first excising the apyrase cDNA insert from the pT7T3D-PAC plasmid using NotI and EcoRI restriction endonucleases. The 1.69 Kb insert was labeled using ³²P-alpha-dATP and the Prime-It II Random Primer Labeling Kit, as directed by the manufacturer (Stratagene). Prehybridization, hybridization, and washes of the membrane were carried out as described by the Master Blot manufacturer (Clontech). The blot was exposed to Kodak BioMax Mr film (Eastman Kodak Co.) for one hour at −80° C. and subsequently developed. For scanning densitometry, the blot was scanned and NIH Imaging Software was used to quantify the signals.

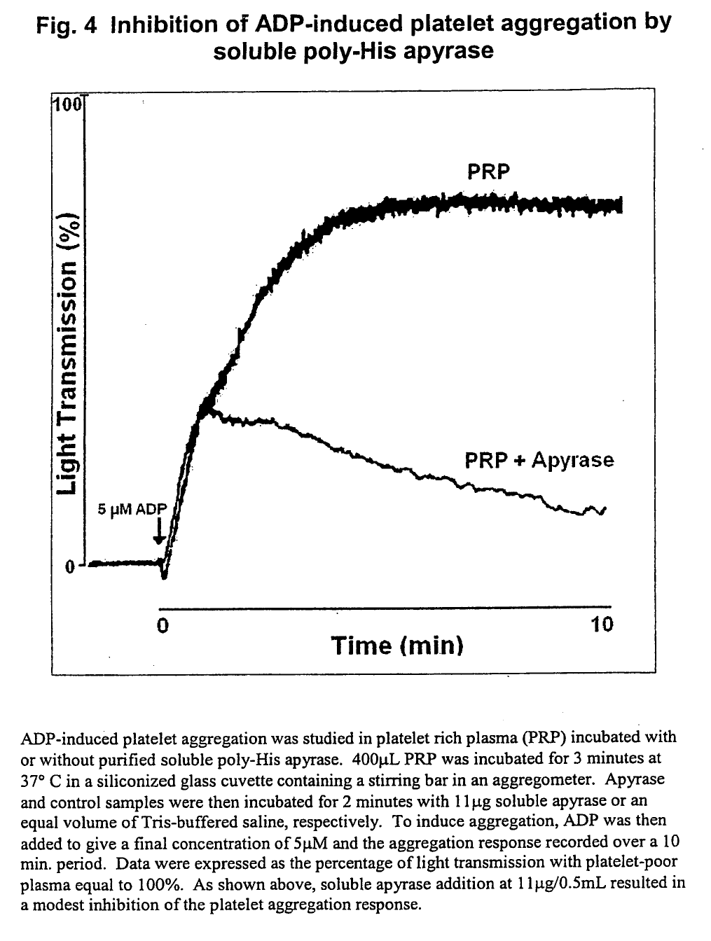

The results are shown in FIGS. 4A and 4B. FIG. 4A is a representation of a dot blot, and FIG. 4B is a summary of relative expression in the indicated tissues. Expression was most abundant in testis, followed by placenta, small intestine, and prostate. mRNA was also detected in lung, stomach, salivary gland, and to a lesser extent, colon. Numbers in parentheses below the 8 major tissues indicate the expression levels relative to testis (100%). the highest levels of mRNA were found in testis, followed by prostate, placenta, and small intestine. Lower levels of mRNA were also detected in lung, stomach, salivary gland, and colon.

### EXAMPLE 3

### Alignment of an AYPR Polypeptide Sequence with Related Polypeptide Sequences

The soluble human apyrase amino acid sequence was aligned with the five most similar sequences in the NCBI protein database (See Table 1 for references to the proteins used in the alignment) using the CLUSTALW alignment algorithm (Thompson et al., NAR 22:4673-80, 1994). The deduced amino acid sequence of the human apyrase cDNA was compared to the non-redundant database of sequences using the basic BLASTP search program available through the NCBI at http://www.ncbi.nlm.nih.gov/BLAST/. The deduced amino acid sequence of the human apyrase was analyzed for putative sorting signals and post-translational modifications using the PSORT II and ScanProsite programs, respectively. Both programs are available through the ExPASy (Expert Protein Analysis System) proteomics server of the Swiss Institute of Bioinformatics at http://www.expasy.ch/. A multiple protein sequence alignment of the human apyrase clone, along with the sequences most closely related to it, was performed using the CLUSTALW program, and the resulting aligned sequences were shaded using BOXSHADE. Both programs are available through the Biology Workbench web site at http://workbench.sdsc.edu/. Unless otherwise indicated, the default analysis parameters were used for all of the computer analyses performed.

The results are shown in FIG. 5. Residues were colored using the BOXSHADE program. Black boxing indicates invariant amino acids, while conserved amino acids are highlighted in gray. Alignment gaps in the sequences are represented by a dash (−). Several strictly conserved acidic amino acids can be found that may represent residues important in the catalytic activity of the enzymes.

Invariant amino acids are found distributed throughout the aligmnent, as well as many highly conserved regions of residues. Although the Cimex lectularius, Phlebotomus papatasi and Lutzomyia longipalpis have known salivary gland apyrase activities, it is not known whether the D. melanogaster or Caenorhabditis elegans proteins have apyrase activity. Based on the sequence similarities observed between the human apyrase and hematophagous insect apyrases with the worm and fruitfly proteins however, it is likely that they do.

### EXAMPLE 4

### An APYR Polypeptide Contains N-Linked Glycans

The glycosylation state of APYR polypeptides was examined by determining their sensitivity to endoglycosidase digestion. Glycosylation status was assessed using antibodies raised against regions of the AYPR polypeptide.

Anti-peptide polyclonal antiserum was generated in rabbits by BioSource International, Inc. (Hopkinton, Mass.) using a synthetic peptide designed from a carboxy-terminal amino acid sequence. The peptide sequence synthesized (C—LPETKIGSVKYEGIEFI—COOH) (SEQ ID NO:10) represents amino acids 317-333 of the apyrase protein. The synthetic peptide was both generated and coupled to keyhole limpet agglutinin by BioSource International, Inc. An affinity column that contained the synthesized peptide was generated by Biosource International and used to affinity purify the antibodies from the serum.

For removal of N-linked glycans, 50 μL of the concentrated media from mock transfected and soluble apyrase transfected COS cells were acetone precipitated [Hager et al., Anal. Biochem. 109:76-86, 1980] and dissolved in 0.2% SDS. After sonication and boiling for 3 minutes, the SDS concentration was adjusted to 0.1% by the addition of reaction buffer containing peptide N-glycosidase F [Treuheit et al., J. Biol. Chem. 267:11777-11782, 1992]; Tarentino et al., J. Biol. Chem. 24:4665-4671, 1985]. The deglycosylation reaction was incubated at 37° C. overnight and subsequently analyzed by SDS-PAGE.

SDS-PAGE was performed following the method of Laemmli [Laemmli, Nature, 227:680-685, 1970]. Control or soluble apyrase transfected COS-1 cell conditioned media were boiled for 5 minutes in SDS sample buffer containing 30 mM dithiothreitol and 8 M urea and were resolved on a 4 to 15% linear gradient SDS-polyacrylamide gel. After electrophoresis, the separated proteins were transferred to poly(vinylidene difluoride) (PVDF) membrane by electroblotting for 3 hours at 33 V in cold 10 mM CAPS (pH 11) [Matsudaira, J. Biol. Chem. 262:10035-10038, 1987]. After transfer, the PVDF membrane was blocked for 1 hour in 5% nonfat dry milk in Tris-buffered saline containing 0.02% sodium azide and incubated overnight at room temperature in a 1 ug/ml dilution of affinity-purified polyclonal antibodies in blocking buffer. The PVDF membranes were subsequently washed, incubated in goat anti-rabbit IgG horseradish peroxidase secondary antibody, and washed again, and immunoreactivity was detected by chemiluminescence with Amersham ECL reagents as described by the manufacturer.

50 μL of the concentrated media from mock transfected and soluble apyrase transfected COS cells were acetone precipitated and deglycosylated at 37° C. overnight as described in the methods. Apyrase protein was detected using the affinity purified anti-peptide antibody against the soluble apyrase. In the conditioned media of apyrase transfected COS cells, the apyrase protein shifts in molecular weight after deglycosylation, indicating that the protein contains N-linked glycans. No such apyrase protein was detected in the media of mock-transfected cells.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A purified polypeptide comprising an amino acid sequence at least 80% identical to amino acids 39-371 of SEQ ID NO:2.

2. The polypeptide of claim 1, wherein the polypeptide hydrolyzes adenosine triphosphate (ATP) or adenosine diphosphate (ADP).

3. The polypeptide of claim 2, wherein the ATP or ADP hydrolytic activity of the polypeptide is calcium-dependent.

4. The polypeptide of claim 2, wherein the ATP or ADP hydrolytic activity of the polypeptide is insensitive to sodium azide.

5. The polypeptide of claim 1, wherein the polypeptide comprises at least one amino acid sequence selected from the group consisting of IADLD (SEQ ID NO:3), DDRTG (SEQ ID NO:4), PWVIL (SEQ ID NO:5), GFKAEW (SEQ ID NO:6), FLPR (SEQ ID NO:7), NIGF (SEQ ID NO:8), and KYEG (SEQ ID NO:9).

6. The polypeptide of claim 1, wherein the polypeptide comprises at least three amino acid sequences selected from the group consisting of IADLD (SEQ ID NO:3), DDRTG (SEQ ID NO:4), PWVIL (SEQ ID NO:5), GFKAEW (SEQ ID NO:6), FLPR (SEQ ID NO:7), NIGF (SEQ ID NO:8), and KYEG (SEQ ID NO:9).

7. The polypeptide of claim 1, wherein the polypeptide comprises the amino acid sequences IADLD (SEQ ID NO:3) and KYEG (SEQ ID NO:9).

8. A purified polypeptide comprising an amino acid sequence at least 95% identical to amino acids 39-371 of SEQ ID NO:2 wherein the polypeptide hydrolyzes adenosine triphosphate (ATP) or adenosine diphosphate (ADP).

9. The purified polypeptide of claim 8 wherein the polypeptide comprises at least three amino acid sequences selected from the group consisting of IADLD (SEQ ID NO:3), DDRTG (SEQ ID NO:4), PWVIL (SEQ ID NO:5), GFKAEW (SEQ ID NO:6), FLPR (SEQ ID NO:7), NIGF (SEQ ID NO:8), and KYEG (SEQ ID NO:9).

10. A purified polypeptide comprising amino acids 39-371 of SEQ ID NO:2.

11. A purified polypeptide consisting of amino acids 39-371 of SEQ ID NO:2.

12. An isolated nucleic acid molecule encoding the polypeptide of claim 1.

13. The nucleic acid molecule of claim 12, wherein the nucleic acid encodes a polypeptide comprising an amino acid sequence at least 95% identical to amino acids 39-371 of SEQ ID NO:2.

14. The nucleic acid molecule of claim 12, wherein the nucleic acid molecule comprises a nucleic acid sequence at least 95% identical to nucleic acids 606-1604 of SEQ ID NO:1.

15. An oligonucleotide at least 10 nucleotides in length that includes at least nine contiguous nucleotides of SEQ ID NO:1.

16. An isolated nucleic acid molecule comprising a nucleotide sequence, which is substantially complementary to a nucleotide sequence of claim 12.

17. The isolated nucleic acid molecule of claim 16 wherein the nucleotide sequence comprises at least 20 nucleotides.

18. An isolated nucleic acid molecule comprising a strand of nucleotides that hybridizes to a polynucleotide complementary to a nucleotide sequence of claim 11.

19. An isolated nucleic acid molecule of claim 18, wherein the length of nucleic acid molecule consists of 20 to 100 nucleotides.

20. A fusion protein comprising a first polypeptide according to claim 1 operably linked to a second polypeptide, there being a cleavage site between said polypeptides.

21. An isolated and purified nucleic acid molecule, which comprises a nucleotide sequence coding for a fusion protein according to claim 20.

22. A vector comprising the nucleic acid of claim 13.

23. A host cell that includes the vector of claim 22.

24. A process for producing a polypeptide comprising culturing the host cell of claim 23 under conditions sufficient for the production of the polypeptide, and recovering the peptide from the host cell culture.

25. A vector according to claim 22, wherein the vector is selected from the group consisting of a plasmid, virus, and bacteriophage.

26. A vector according to claim 22, wherein the isolated nucleic acid molecule is inserted into the vector in proper orientation and correct reading frame such that a cell transformed with the vector may express the protein of SEQ ID NO:2.

27. A vector according to claim 26, wherein the isolated nucleic acid molecule is operatively linked to a promoter sequence.

28. A nucleic acid construct comprising the nucleic acid sequence of claim 1 operably linked to one or more control sequences that direct the production of the polypeptide in a suitable expression host.

29. A recombinant expression vector comprising the nucleic acid construct of claim 28, a promoter, and transcriptional and translational stop signals.

30. A recombinant host cell comprising the vector of claim 29.

31. A recombinant host cell of claim 30 wherein the host cell is a mammalian cell.

32. A process for producing a polypeptide comprising culturing the host cell of claim 31 under conditions sufficient for the production of the polypeptide, and recovering the peptide.

33. An antibody that selectively binds the polypeptide of claim 1.

34. A pharmaceutical composition comprising the polypeptide of claim 1, and a pharmaceutically acceptable carrier.

35. A pharmaceutical composition comprising the polynucleotide of claim 13, and a pharmaceutically acceptable carrier.

36. A pharmaceutical composition comprising the vector of claim 22, and a pharmaceutically acceptable carrier.

37. A method of producing a polypeptide, the method comprising culturing a cell comprising the nucleic acid of claim 13 under conditions allowing for expression of the polypeptide encoded by the nucleic acid.

38. A method for identifying a modulator of platelet aggregation, the method comprising (a) contacting a test agent with the polypeptide of claim 1; and (b) determining whether the test agent binds specifically to the polypeptide, thereby identifying a modulator of platelet aggregation.

39. A method of hydrolyzing ATP or ADP in a biological sample, the method comprising contacting the sample with the polypeptide of claim 1.

40. The method of claim 39, wherein the sample comprises a blood cell.

41. The method of claim 39, wherein the sample is provided in vitro.

42. The method of claim 39, wherein the sample is provided in vivo.

43. A method of inhibiting platelet aggregation in a subject, the method comprising introducing into the subject an agent that increases levels of the polypeptide of claim 1 in the subject.

44. The method of claim 43, wherein the subject is a human.

45. The method of claim 43, wherein the agent is a polypeptide comprising amino acids 39-371 of SEQ ID NO:2.

46. The method of claim 43, wherein the agent is a polynucleotide encoding a polypeptide comprising amino acids 39-371 of SEQ ID NO:2.

47. A method of treating a thrombosis in a subject, the method comprising introducing into the subject an agent that increases levels of the polypeptide of claim 1 in the subject.

48. A method of assaying for compounds which bind to an apyrase protein, which comprises (a) contacting a polypeptide derivative according to claim 1 or 5 with a compound to be investigated; and (b) detecting whether the compound binds to the polypeptide derivative.

49. A method according to claim 48 wherein the compound to be investigated is an antibody.

50. The antibody of claim 33 wherein the antibody neutralizes ATP or ADP hydrolytic activity of an AYPR polypeptide.


SEQUENCE LISTING (20 sequences)

SEQ ID NO: 1 - DNA, 1691 bp, Homo sapiens
  CDS (492)...(1607)
  sig_peptide (605)...(605)
  N_region (664)...(673)
  misc_feature (723)...(725): Putative Asn-glycan
ggcacgaggggcggaagtcggccaccttcctccgtcccggccgttagcccagccaagcccagccaagcccagccaagccccgccgatcgcgggcaccggagccagccccgcagcgggagcctaacccagaggaagtggagactcctttactgcacatctgtctgctccaggccaggcactgttctaggagccaggatacagaggtggagacagaaacaaacaaaccgataactgtatacaatactctaacgtcagagggaccacagagcaccagcctgcatggaacttccttcctcactcagcttcccacgttgccagctgggacaggggagatggagtaattttgctgtggaaagacttcacgtcttgccgaatgaaagtcccgcctgtctgtcacgctgatgcccgtgcagctgtctgagcacccggaatggaatgagtctatgcactccctccggatcagtgtggggggccttcctgtgctggcgtccatgaccaaggccgcggacccccgcttccgcccccgctggaaggtgatcctgacgttctttgtgggtgctgccatcctctggctgctctgctcccaccgcccggcccccggcaggccccccacccacaatgcacacaactggaggctcggccaggcgcccgccaactggtacaatgacacctaccccctgtctcccccacaaaggacaccggctgggattcggtatcgaatcgcagttatcgcagacctggacacagagtcaagggcccaagaggaaaacacctggttcagttacctgaaaaagggctacctgaccctgtcagacagtggggacaaggtggccgtggaatgggacaaagaccatggggtcctggagtcccacctggcggagaaggggagaggcatggagctatccgacctgattgttttcaatgggaaactctactccgtggatgaccggacgggggtcgtctaccagatcgaaggcagcaaagccgtgccctgggtgattctgtccgacggcgacggcaccgtggagaaaggcttcaaggccgaatggctggcagtgaaggacgagcgtctgtacgtgggcggcctgggcaaggagtggacgaccactacgggtgatgtggtgaacgagaaccccgagtgggtgaaggtggtgggctacaagggcagcgtggaccacgagaactgggtgtccaactacaacgccctgcgggctgctgccggcatccagccgccaggctacctcatccatgagtctgcctgctggagtgacacgctgcagcgctggttcttcctgccgcgccgcgccagccaggagcgctacagcgagaaggacgacgagcgcaagggcgccaacctgctgctgagcgcctcccctgacttcggcgacatcgctgtgagccacgtcggggcggtggtccccactcacggcttctcgtccttcaagttcatccccaacaccgacgaccagatcattgtggccctcaaatccgaggaggacagcggcagagtcgcctcctacatcatggccttcacgctggacgggcgcttcctgttgccggagaccaagatcggaagcgtgaaatacgaaggcatcgagttcatttaactcaaaacggaaacactgagcaaggccatcaggactcagcttttataaaaacaagaggagtgcaaaaaaaaaaaaaaaaaaaaa

SEQ ID NO: 2 - protein, 1116 aa, Homo sapiens
ATGACCAAGGCCGCGGACCCCCGCTTCCGCCCCCGCTGGAAGGTGATCCTGACGTTCTTTGTGGGTGCTGCCATCCTCTGGCTGCTCTGCTCCCACCGCCCGGCCCCCGGCAGGCCCCCCACCCACAATGCACACAACTGGAGGCTCGGCCAGGCGCCCGCCAACTGGTACAATGACACCTACCCCCTGTCTCCCCCACAAAGGACACCGGCTGGGATTCGGTATCGAATCGCAGTTATCGCAGACCTGGACACAGAGTCAAGGGCCCAAGAGGAAAACACCTGGTTCAGTTACCTGAAAAAGGGCTACCTGACCCTGTCAGACAGTGGGGACAAGGTGGCCGTGGAATGGGACAAAGACCATGGGGTCCTGGAGTCCCACCTGGCGGAGAAGGGGAGAGGCATGGAGCTATCCGACCTGATTGTTTTCAATGGGAAACTCTACTCCGTGGATGACCGGACGGGGGTCGTCTACCAGATCGAAGGCAGCAAAGCCGTGCCCTGGGTGATTCTGTCCGACGGCGACGGCACCGTGGAGAAAGGCTTCAAGGCCGAATGGCTGGCAGTGAAGGACGAGCGTCTGTACGTGGGCGGCCTGGGCAAGGAGTGGACGACCACTACGGGTGATGTGGTGAACGAGAACCCCGAGTGGGTGAAGGTGGTGGGCTACAAGGGCAGCGTGGACCACGAGAACTGGGTGTCCAACTACAACGCCCTGCGGGCTGCTGCCGGCATCCAGCCGCCAGGCTACCTCATCCATGAGTCTGCCTGCTGGAGTGACACGCTGCAGCGCTGGTTCTTCCTGCCGCGCCGCGCCAGCCAGGAGCGCTACAGCGAGAAGGACGACGAGCGCAAGGGCGCCAACCTGCTGCTGAGCGCCTCCCCTGACTTCGGCGACATCGCTGTGAGCCACGTCGGGGCGGTGGTCCCCACTCACGGCTTCTCGTCCTTCAAGTTCATCCCCAACACCGACGACCAGATCATTGTGGCCCTCAAATCCGAGGAGGACAGCGGCAGAGTCGCCTCCTACATCATGGCCTTCACGCTGGACGGGCGCTTCCTGTTGCCGGAGACCAAGATCGGAAGCGTGAAATACGAAGGCATCGAGTTCATTTAA

SEQ ID NO: 3 - protein, 403 aa, Homo sapiens
MPIQPFDQREWNEPMHSLRISVGGLPVLASMTKATDPRFRPRWRVILTSFVGAALLWLLYSHHQTPVSGRPPIHNAHNWRLRQERISQYNDTYPLSPPQRTPGGIRYRIAVIADLDTGSKAQEENTWFSYLKKGYLTLSDSGDRVSVEWDKDRGVLESHLAEKGRGMELSDLIVFNGKLYSVDDRTGVIYQIEGTKAVPWVILSDGDGAVEKGFKAEWLAVKDEHLYVGGLGKEWTTTTGEVVNENPEWVKVVGHRGSVEHENWVSSYNALRAAAGIQPPGYLIHESACWSDTLQRWFFLPRRASHERYSEREDERKGSNLLLSAAQDFRDISVRQVGALVPTHGFSSFKFIPNTDDQIIVALKSEEDNGRIATYVMAFTLDGRFLLPETKIGSVKYEGIEFI

SEQ ID NO: 4 - protein, 403 aa, Homo sapiens
MPIQPFDQREWNEPMHSLRISVGGLPVLASMTKATDPRFRPRWRVILTSFVGAALLWLLYSHHQGPVPGRPPTHNAHNWRLSQQRISHYNDTYPLSPPQRTPGGIRYRIAVIADLDTGSRAQEENTWFSYLKKGYLTLSDSGDRVSVEWDKDHGVLESHLAEKGRGMELSDLIVFNGKLYSVDDRTGVIYQIEGTKAVPWVILSDGDGTVEKGFKAEWLAVKDEHLYVGGLGKEWTTTTGEVMNENPEWVKVVGHRGSVDHENWVSSYNALRAAAGIRPPGYLIHESACWSDTLQRWFFLPRRASHERYSEKDDERKGSNLLLSAAQDFRDISVRQVGTLIPTHGFSSFKFIPNTDDQIIVALKSEEDNGRIATYVMAFTLDGRFLLPETKIGTVKYEGIEFI

SEQ ID NO: 5 - protein, 371 aa, Homo sapiens
MTKAADPRFRPRWKVILTFFVGAAILWLLCSHRPAPGRPPTHNAHNWRLGQAPANWYNDTYPLSPPQRTPAGIRYRIAVIADLDTESRAQEENTWFSYLKKGYLTLSDSGDKVAVEWDKDHGVLESHLAEKGRGMELSDLIVFNGKLYSVDDRTGVVYQIEGSKAVPWVILSDGDGTVEKGFKAEWLAVKDERLYVGGLGKEWTTTTGDVVNENPEWVKVVGYKGSVDHENWVSNYNALRAAAGIQPPGYLIHESACWSDTLQRWFFLPRRASQERYSEKDDERKGANLLLSASPDFGDIAVSHVGAVVPTHGFSSFKFIPNTDDQIIVALKSEEDSGRVASYIMAFTLDGRFLLPETKIGSVKYEGIEFI

SEQ ID NO: 6 - protein, 296 aa, Homo sapiens
MLITDLDHDSKDGKKWKSLVSRGFLKVSADHKHADIHFDKDSEYYVDTNIAAGGRAMELSDLAVFNGKLYSIDDRTGLIYQISDKKALPWVLLNDGPGNVVKGFKGEWITVKDTELIVGGLGKEWTTTDGVYVNDHPMWVKHVSAHGAVHHENWKDVYIRVRRAAGIEYPGYMIHEAVQWSAIHRKWFFLPRRMSNEKYSEAEDENRGTNVLVIGNEELTDFEVVRVGSENNKSRGFAAFQFVPNTHHQLIVAIKSEEKDGKPVASYASVFDIHGNVILDEYLLHGPYKYEGIAFA

SEQ ID NO: 7 - protein, 392 aa, Homo sapiens
MKQFNGSISPSSSSSSLPFASLGSGKTSSKQSSGSAIIDTGMYLRDWRKALRSPPSYRIGNRTIRLQVHFTWVLAALCAFLLLVLYISSSPSSLLSDGPRTNSFLRTSAIVYNHTYPLTSPIVSSGIYSFRVGIIADLDTNSALKKNQWGSYYLKGCLSFIPSKRSITVSWDEGEAKALQSGFALKGRGMELSELVVFNGKLLTFDDRTGLVYEIEGEKVIPWVLLMDGDGRTSKGFKSEWATVKDQVLYVGSMGKEWTTSAGDFETHDPMYVKAVTVHGEVYHLTWINHYKAIRKAIGIEWPGYMIHESGAWSEVHRRWFFLPRRCSRARYNETRDEHMGCNFLISSDETFQNIRAIELKRNDVPATHGYSSFKFLPTSNDEIIVALSTES

SEQ ID NO: 8 - protein, 419 aa, Homo sapiens
MQSPAFAYREHAEHSPRAMYMRDWRSALRTPTYRIGNRTVRFNYHFALLIVCAFVLVLLFYFARQGSHSSSDGYWLRRSYTDARSLDNGGPVVQAYNATYPLTSPMVLRGGVINYRIAMIADLDTSSKVSKGDGSSTWRSYLKKGYLTYTVARSEIQISWDDGAPIVLESAFALKGRGMELSELVTFNGRLLTFDDRTGLIYEIVNDKPIPWVILLDGDGHSAKGFKAEWATVKEQTLYVGSMGKEWTTSAGDFENNNPMYVKAITPSGEVRSLNWVDNFKQLRLQSMQITWPGYMIHESGTWSEERNRWFFLPRRCSKEKYNETKDEHMGCNVLVSADESFTNVETVRLDPENTTPTHGFSSFKFLPGTDDSIIVALKSEELNGKTATFITAFDIAGKTLLPETRIETDYKYEGFEFI

SEQ ID NO: 9 - protein, 364 aa, Homo sapiens
MRSSYRVGNPIRFQPTNVVGLLLLSLVLSFMLVQSYELGHASGETNANSKYPLTTPVEENLKVRFKIGVISDDDKNAVSKDESNTWVSTYLTGTLEWEKSTDKITVQWDKGNEKKVKSKYSYGGRGMELSELVTFNGNLLTFDDRTGLVYILKDDKVYPWVVLADGDGKNSKGFKSEWATEKAGNLYVGSSGKEWTTKEGTIENYNPMWVKMINKNGEVTSLNWQTNYEKIRSSMNITFPGYMWHEAACWSDKYNKWFFLPRALSQEAYDSKKFETQGANVIISCDDKFEKCEPTQIQGKTEDKRGFSNFKFVPTSEDKIIVGLKTVEADDTTETYFTAFDLEGKVLLEETKIDDHKYEGVDFV

SEQ ID NO: 10 - protein, 325 aa, Homo sapiens
MFLKWVVCAFATVFLVGVSQAAPPGVEWYHFGLIADMDKKSIASDKTTFNSVLKIDELRHNTKTDQYIYVRSRVKKPVSTRYGFKGRGAELSEIVVFNNKLYTVDDKSGITFRITKDGKLFPWVILADADGQRPDGFKGEWATIKDDTIYVGSTGMLKFTSSLWVKKITKDGVVTSHDWTDKYRKILKALNMPNGFVWHEAVTWSPFRKQWVFMPRKCSRHPFSQELEERTGCNKIVTADENFNDIQVIHIQDQPYNLASGFSSFRFIPGTKNERLLALRTVEQEDQVKTWAVVMDMKGTVLMYEKELYDEKFEGLAFFGGIKKN

SEQ ID NO: 11 - protein, 336 aa, Homo sapiens
MFLKFCIVAFAICLSINLSEGAPRSGTIYNFAIIADLDKKSISPKNDNNYKSIVKVGELIEVGDKYSVKMKKEDHEIFTKYAYKGRGAELSEFLIYKWKLYTFDDKSGIVFRLKTNADLIPWVTLANGNGDQTDGFKAEWATTKGDKMYVGSTGISFTDKTGKLNSNSLWIKEIDQDGKVQSLDWKEQYDKIKSAMKIPNGFIWHEAVNWSKLKNQWVFLPRKCSERPFDTKTEETIGCNKIIIASENFEIIKSIQIKGKSINRAAGFSSFKFLPDSDDQILLALKTIEKDDKTATYITVIDITGRVLMPEMQINSDKYEGIVLLKSTEGFLKRSQ

SEQ ID NO: 12 - protein, 5 aa, Homo sapiens
IADLD

SEQ ID NO: 13 - protein, 5 aa, Homo sapiens
DDRTG

SEQ ID NO: 14 - protein, 5 aa, Homo sapiens
PWVIL

SEQ ID NO: 15 - protein, 6 aa, Homo sapiens
GFKAEW

SEQ ID NO: 16 - protein, 4 aa, Homo sapiens
FLPR

SEQ ID NO: 17 - protein, 4 aa, Homo sapiens
NIGF

SEQ ID NO: 18 - protein, 4 aa, Homo sapiens
KYEG

SEQ ID NO: 19 - protein, 17 aa, Homo sapiens
LPETKIGSVKYEGIEFI

SEQ ID NO: 20 - protein, 333 aa, Homo sapiens
PPTHNAHNWRLGQAPANWYNDTYPLSPPQRTPAGIRYRIAVIADLDTESRAQEENTWFSYLKKGYLTLSDSGDKVAVEWDKDHGVLESHLAEKGRGMELSDLIVFNGKLYSVDDRTGVVYQIEGSKAVPWVILSDGDGTVEKGFKAEWLAVKDERLYVGGLGKEWTTTTGDVVNENPEWVKVVGYKGSVDHENWVSNYNALRAAAGIQPPGYLIHESACWSDTLQRWFFLPRRASQERYSEKDDERKGANLLLSASPDFGDIAVSHVGAVVPTHGFSSFKFIPNTDDQIIVALKSEEDSGRVASYIMAFTLDGRFLLPETKIGSVKYEGIEFI